(19) United States
(12) Patent Application Publication
(10) Pub. No.: US 2025/0200930 A1
(43) Pub. Date: Jun. 19, 2025

(54) Automatic Inner, Middle and Outer Ear Segmentations

(71) Applicant: CASCINATION AG, Bern (CH)

(72) Inventors: Daniel Erpenbeck, Bern (CH); Sergii Poltaretskyi, Bern (CH)

(21) Appl. No.: 18/843,298

(22) PCT Filed: Mar. 2, 2023
(86) PCT No.: PCT/IB2023/051969

§ 371 Date: Sep. 2, 2024

(30) Foreign Application Priority Data
Mar. 2, 2022 (EP) 22159818.8

(51) Int. Cl. G06V 10/26; G06V 10/74; G06V 10/75; G06V 40/10

(52) U.S. Cl. CPC G06V 10/26; G06V 10/7553; G06V 10/761; G06V 40/10; G06V 2201/03

(57) ABSTRACT

The present invention relates to a method for automatically segmenting the cochlea of a person comprised in an image (M) comprised of voxels (v) each voxel comprising an intensity value being indicative of the intensity of the respective voxel (v).

### PRIOR RELATED APPLICATIONS

This application claims the benefit of priority of prior-filed European Patent Application No. 22159818.8, filed Mar. 2, 2022.

### SPECIFICATION

The present invention relates to a method for detecting and segmenting the cochlea of a person as well as to a corresponding system, and computer program.

The inner ear (auris interna) is the innermost part of the human ear and comprises two important functional part, the cochlea needed for converting sound pressure from the outer ear into electrochemical impulses being passed to the brain via the auditory nerve for sound perception, as well as the vestibular system as a means to maintain to balance. Due to the fact that these structures are rather tiny in size, surgery performed on these organs needs to be carried out with utmost precision.

Typical surgeries performed on the human ear are acoustic neuroma surgery for removing an acoustic neuroma, particularly by one of translabyrinthine, wherein the mastoid bone and bone in the inner ear are removed for access to the ear canal to remove the tumor; retrosigmoid/suboccipital, wherein the surgeon makes an incision through an opening in the skull, behind the mastoid part of the ear; middle fossa, wherein the surgeon removes the tumor from the upper surface of the internal ear canal beyond the inner ear.

Another typical surgery is the cochlear implant (CI) ear surgery. Such a surgically-implanted electronic device helps to restore sound perception to people with severe hearing loss caused e.g. by a damage or a defect in the inner ear. Particularly, such CI can be configured to directly stimulate the auditory nerve to send information to the brain.

Particularly, in the natural hearing process, ear anatomy mechanically translates sound into vibrations of the basilar membrane in the cochlea, which stimulate nerves connected to the spiral ganglion and, eventually, the auditory nerve, wherein higher frequencies cause stimulation of more basal spiral ganglion nerves, while lower frequencies stimulate more apical spiral ganglion nerves. Thus, a CI can be configured to use this so-called natural tonotopy by applying an electric field to more apical (basal) spiral ganglion nerves to induce perceived lower (higher) frequency sounds.

In this way, CIs induce hearing sensation by stimulating auditory nerve pathways within the cochlea using e.g. an implanted electrode array. A processor of the CI can be programmed to process sound detected by a microphone and to send signals to each electrode. CI electrode arrays are configured such that when properly positioned, each electrode stimulates nerve pathways corresponding to a pre-defined frequency bandwidth. However, in surgery, the CI electrode array is blindly threaded into the cochlea with its insertion path guided only by the walls of the spiral-shaped intra-cochlear cavities. Since the final positions of the electrodes are generally unknown, the only option when programming the CI is to assume the electrodes are optimally positioned in the cochlea and use a default frequency allocation table.

Yet another surgical procedure is the congenital atresia ear reconstruction, particularly aiming to reconstruct parts such as the ear canal, tympanic membrane (eardrum), ossicular chain (middle ear bones of hearing).

Furthermore, implantation of an osseointegrated bone conduction hearing system allows to surgically place a bone anchored hearing aid to the skull to transmit sound through the bone to the inner ear.

Furthermore, using procedures such as stapedectomy/stapedotomy the stapes bone can be removed and replace it with a prosthesis.

Yet another surgical procedure, namely tympanoplasty, can be used on the eardrum and/or middle ear bones to restore the middle ear hearing mechanism.

All implantation/surgical procedures outlined above benefit greatly from a precise knowledge of the location and shape of the individual part of the ear.

Therefore, the problem to be solved by the present invention therefore is to provide a method, a system and a computer program for reliably detecting and segmenting the cochlea of a person.

This problem is solved by a method having the features of claim 1, a system having the features of claim 17, and a computer program having the features of claim 18.

Preferred embodiments of these aspects of the present invention are stated in the corresponding dependent claims and are described below.

According to claim 1 a method for automatically segmenting the cochlea of a person comprised in an image M comprised of voxels v is disclosed, each voxel comprising an intensity value being indicative of the intensity of the respective voxel v, the method comprising the steps of:
Providing an empty first gradient accumulator image E₁ comprised of voxels v_{E1} having the same origin and size as the image M, each voxel v_{E1} of the first gradient accumulator image E₁ being associated with a voxel v of the image M, and computing for each voxel v of the image M a gradient vector g pointing in a direction towards a largest possible intensity increase of the image M as well as a first gradient line extending in the direction of the gradient vector g, the first gradient line starting at a first start point p_{start,1} and ending at a first end point p_{end,1}, wherein the intensity value of each voxel v_{E1} of the first gradient accumulator image E₁ whose corresponding voxel v of the image M intersects with the respective first gradient line is increased by a pre-defined constant increment,
Providing an empty second gradient accumulator image E₂ comprised of voxels v_{E2} having the same origin and size as the image M each voxel v_{E2} of the second gradient accumulator image E₂ being associated with a voxel v of the image M, and computing for each voxel v of the image M a gradient vector g pointing in a direction towards a largest possible intensity increase of the image M as well as a second gradient line extending in the opposite direction of the gradient vector g, the second gradient line starting at a second start point p_{start,2} and ending at a second end point p_{end,2}, wherein the intensity value of each voxel v_{E2} of the second gradient accumulator image E₂ whose corresponding voxel v of the image M intersects with the respective second gradient line is increased by a pre-defined constant increment,
Providing an empty third gradient accumulator image E₃ comprised of voxels v_{E3} having the same origin and size as the image M each voxel v_{E3} of the third gradient accumulator image E₃ being associated with a voxel v of the image M, and computing for each voxel v of the image M a gradient vector g pointing in a direction towards a largest possible intensity increase of the image M as well as a third gradient line extending in the direction of the gradient vector g, the third gradient line starting at a third start point p_{start,3} and ending at a third end point p_{end,3}, wherein the intensity value of each voxel v_{E3} of the first gradient accumulator image E₃ whose corresponding voxel v of the image M intersects with the respective third gradient line is increased by a pre-defined constant increment, and
forming a cochlea descriptor image E_{descriptor} comprised of voxels v_{c} using the first, second and third gradient accumulator images E₁, E₂, E₃, and selecting a number P≥1 of voxels v_{c} of the cochlea descriptor image E_{descriptor} having intensity values being larger than the intensity values of all other voxels of the cochlea descriptor image E_{descriptor} as predictions of possible locations of the center of the cochlea in said image M.

Particularly, according to an embodiment of the present invention, said selecting of said number P≥1 of voxels v_{c} comprises thresholding the cochlea descriptor image E_{descriptor} with a value obtained from multiplying the brightest voxel (i.e. highest intensity) of the descriptor image with a factor in the range of 0.6 to 0.8, wherein preferably the factor is 0.7. The thresholding will create one or several 3D objects comprising the highest accumulated densities, wherein each of said voxels v_{c} corresponds to a center of such an object. This strategy is advantageous since the majority of cochlea gradients will not converge to one voxel, but rather several neighboring voxels. So, in order to reinforce the accuracy of the detection (especially on noisy images) one preferably searches for objects with highest intensity.

According to an embodiment of the method, forming the cochlea descriptor image corresponds to computing the Hadamard product (E₁° E₂° E₃)ᵢⱼ=(E₁)ᵢⱼ(E₂)ᵢⱼ(E₃)ᵢⱼ of the first, second and third gradient accumulator images (E₁, E₂, E₃).

Further, according to an embodiment of the method, the respective gradient line comprises a length extending from the respective start point to the respective end point, wherein the length of each first gradient line is larger than the length of each third gradient line, and wherein the length of each third gradient line is larger than the length of each second gradient line.

Further, according to an embodiment of the method, the respective first, second and third start point p_{start,1}, p_{start,2}, p_{start,3} is computed according to

$${p}_{start , i} = position ( {v}_{M} ) + {start}_{i} \cdot g \qquad i = 1 , 2 , 3$$

and wherein the respective first, second and third end point p_{end,1}, p_{end,2}, p_{end,3} is computed according to

$${p}_{end , i} = position ( {v}_{M} ) + {end}_{i} \cdot g \qquad i = 1 , 2 , 3$$

wherein position(v_{M}) denotes the position of the receptive voxel (v) of the image (M).

Furthermore, according to an embodiment of the method, start₁ is in the range from 2.7 mm to 3.3 mm, wherein particularly start₁ is equal to 3.0 mm,

Furthermore, according to an embodiment of the method, start₂ is in the range from −3.3 mm to −2.7 mm, wherein particularly start₁ is equal to −3.0 mm,

Furthermore, according to an embodiment of the method, start₃ is in the range from 0.45 mm to 0.55 mm, wherein particularly start₃ is equal to 0.5 mm,

Furthermore, according to an embodiment of the method, end₁ is in the range from 4.5 mm to 5.5 mm, wherein particularly end₁ is equal to 5.0 mm,

Furthermore, according to an embodiment of the method, end₂ is in the range from −1.8 mm to −2.2 mm, wherein particularly end₂ is equal to −2.0 mm,

Furthermore, according to an embodiment of the method, end₃ is in the range from 1.8 mm to 2.2 mm, wherein particularly end₃ is equal to 2.0 mm.

Furthermore, in an embodiment, said constant increment corresponds to the natural number 1.

Furthermore, according to an embodiment of the method, the respective gradient vector is a normalized gradient vector.

Further, according to an embodiment of the method, the image (M) is one of: a CT image, an MRI image, a cone-beam CT image.

Further, according to an embodiment of the method, the method further comprises the step of:
Providing a statistical shape model of a human ear comprising at least a cochlea part corresponding to the cochlea of the human ear.

Particularly, according to an embodiment of the present invention, besides the cochlea, the SSM comprises at least one of, several of or all of the following further parts of the ear: the three semicircular canals, stapes, incus, malleus, tympanic membrane Statistical shape models (SSM) are known in image analysis. In the context of the present invention the SSM is particularly used for segmentation of the image of the cochlea.

The SSM comprises/describes a mean shape of at least the cochlea (and particularly of the other part(s) present in the SSM), and is indicative of variations of the training datasets with respect to the mean shape. Particularly, according to an embodiment of the present invention, the mean shape is computed from at least 20 training datasets each training dataset corresponding to a 3D image of an ear of a different person, the 3D images comprising at least the cochlea part and particularly said other part(s), see above.

Particularly, for computing the variation, rotation, and translation are removed from each training dataset.

Furthermore, particularly, the variations are extracted from the training datasets using principal component analysis (PCA) by computing the covariance matrix from the training dataset matrix containing the training datasets. The eigenvectors corresponding to the eigenvalues of the covariation matrix correspond to the directions and amount of variation seen in the training datasets. The principal modes of variation are computed based on the first N eigenvectors and associated eigenvalues, where the number N depends on the desired cumulative variance, i.e., the value of variation that can be accounted for by the first N eigenvectors. Possible shapes x of the SSM can then be obtained as a weighted linear combination x=x̅+Ψw of these eigenvectors Ψ around the mean shape x̅, wherein the corresponding weights w are denoted as feature weights.

According to yet another embodiment, the method further comprises the step of:
for each voxel (v_{c}) of said number P of voxels:
   defining a volume in the image (M) having the respective voxel (v_{c}) as a center,
   Segmenting the cochlea within the volume with help of a Hessian-based enhancement filter thereby obtaining a segmented cochlea region of said image M, said region corresponding to the cochlea in the image M, Particularly, the Hessian-based filter can be configured to detect dark tubular structures, wherein particularly α=0.1, β=0.5, and γ=100. The filter is configured in multi-scale mode with a varying between 0.3 mm to 0.7 mm with 3 steps, e.g., σ=0.3, σ=0.5, and σ=0.7. Particularly, the segmented cochlea region can is converted into a 3D mesh, e.g., by applying a marching cube algorithm (see e.g. William E. Lorensen, Harvey E. Cline: Marching Cubes: A high resolution 3D surface construction algorithm. In: Computer Graphics, Vol. 21, Nr. 4, July 1987) to the segmented cochlea region in order to obtain a 3D surface of the cochlea,
   Fitting of said cochlea part of the statistical shape model to the 3D surface of the cochlea. Particularly, the fitting can comprise testing a range of cochlea sizes, i.e. changing the first mode of variation that is responsible for size (scale), wherein particularly the following values of the first entry of w in x=x̅+Ψw are tested: −3.0, −2.0, −1.0, 0.0, 1.0, 2.0, 3.0, wherein for every generated cochlea a rotation around its center is applied in order to find the best orientation.
   Computing a surface similarity measure (e.g. root-mean-square-difference) being indicative of a similarity between a surface of said cochlea part of the statistical shape model and a surface of the segmented cochlea region of the image (M),
Determining the voxel v_{c} of said number P of voxels as center of the cochlea for which the surface similarity measure fulfils a predefined criterion, wherein particularly the first voxel v_{c} among said number P of voxels is chosen as center of the cochlea for which the surface similarity measure drops below a predefined threshold.

Particularly, segmentation of an image corresponds to classifying if a voxel of an image belongs to certain structure. Particularly, the following Hessian-based enhancement filter can be used in this context:

Further, the respective volume can be cuboidal or spherical volume, but may also comprise any other suitable shape, particularly the volume is a cube having a side length of at least 20 mm.

Further, according to an embodiment of the method, the method further comprises the steps of:
fitting the cochlea part of the statistical shape model to the segmented cochlea region using the determined center of the cochlea, the statistical shape model further comprising a semicircular canal part corresponding to the three semicircular canals of the ear,
Predicting locations of the three semicircular canals in the image (M) as the locations of the three semicircular canals in the semicircular canal part of the statistical shape model,
Defining a volume comprising the predicted locations, and
Segmenting the three semicircular canals within the volume (e.g. with help of a Hessian-based enhancement filter) thereby obtaining a segmented semicircular canal region of the image M particularly corresponding to the three semicircular canals in the image M. Particularly, the used Hessian-based enhancement filter is configured to detect dark tubular structures, wherein particularly α=0.5, β=0.1, γ=100, and a between 0.3 mm to 0.7 mm with 3 steps (e.g., σ=0.3, σ=0.5, and σ=0.7).

Furthermore, the three semicircular canals in the image M can be annotated based on the statistical shape model and its fitting into the segmented cochlear region and the segmented semicircular canal region.

Again, the volume used can be a cuboidal volume, particularly a cube, a spherical volume or any other suitable volume.

Further, according to an embodiment of the method, the method further comprises the step of:
fitting the cochlea part and the semicircular canal part of the statistical shape model to the segmented cochlear region corresponding to cochlea in the image and to the segmented semicircular canal region particularly corresponding to the three semicircular canals in the image M.

Advantageously, this step improves the ear SSM shape fitting to anatomical structures and renders further predictions more accurate.

Further, according to an embodiment of the method, the method further comprises the steps of:
Predicting locations of the incus, malleus and stapes in the image as the locations of incus, malleus and stapes in an ossicles part of the statistical shape model, the ossicles part of the statistical shape model corresponding to incus, malleus, and stapes of the ear,
Defining a volume comprising the predicted locations, and
Segmenting the incus, malleus and stapes within the volume thereby obtaining a segmented ossicles region of the image M corresponding to incus, mallus and stapes in the image M.

Further, according to an embodiment of the present invention, annotating the incus, malleus and stapes in the image is performed based on the statistical shape model and its fitting into the segmented cochlea region, segmented semicircular canal region and segmented ossicles region.

Again, the volume used can be a cuboidal volume, particularly a cube, a spherical volume or any other suitable volume.

Further, according to an embodiment of the method, the method further comprises the step of:
Predicting a location of the tympanic membrane as the location of the tympanic membrane in a tympanic membrane part of the statistical shape model, the tympanic membrane part of the statistical shape model corresponding to the tympanic membrane of the ear,
Defining a volume comprising the predicted location, and
Segmenting the tympanic membrane within the volume (e.g. with help of a Hessian-based enhancement filter) thereby obtaining a segmented tympanic membrane region of the image M corresponding to the tympanic membrane in the image M.
Particularly, this Hessian filter can be configured to detect bright plate like structures, wherein particularly α=0.1, β=1.0, γ=50, and σ between 0.2 . . . 0.4 with 2 steps, e.g. σ=0.2 and α=0.4.

Further, according to an embodiment, annotating the tympanic membrane in the image can be conducted based on the statistical shape model and its fitting into at least the segmented cochlear region, segmented semicircular canal region and segmented tympanic membrane region.

Again, the volume used can be a cuboidal volume, particularly a cube, a spherical volume or any other suitable volume.

Particularly, the tympanic membrane is the end of the external auditory canal. Once it has been segmented, one knows where the auditory canal ends and also its orientation (particularly a best fit plane to the tympanic membrane will suffice predicting the external auditory canal direction)

Particularly, in an embodiment, once the end of the external canal end and its direction are known, a cylinder-shaped region of interest is defined (a shape that best describes the canal). In the defined shape the cortical bone is then segmented that represents the canal. This bone can be segmented with simple thresholding, or with the help of a Hessian filter (configured to bright plate-like structures)

Further, according to an embodiment of the method, the method further comprises the steps of:
Predicting a region of location, an orientation and limit of the external auditory canal based on the segmented tympanic membrane region of the image M corresponding to the tympanic membrane in the image M,
Defining a volume comprising the predicted region,
Segmenting the external auditory canal within the volume (e.g. with help of a Hessian-based enhancement filter) thereby obtaining a segmented external auditory canal region of the image M corresponding to the external auditory canal in the image M.

Furthermore, in an embodiment, annotating the external auditory canal in the image can be performed based on the statistical shape model and its fitting into at least the segmented tympanic membrane region.

Again, the volume used can be a cuboidal volume, particularly a cube, a spherical volume or any other suitable volume.

Further, according to an embodiment of the method, the method further comprises the steps of:
Predicting a location and orientation of the external surface of the temporal bone based on the segmented external auditory canal region of the image M corresponding to the external auditory canal in the image M
Defining a volume comprising the predicted location, and
Segmenting the temporal bone within the volume with the help of thresholding thereby obtaining a segmented temporal bone region of the image (M) comprising the external surface of the temporal bone (and particularly an adjacent layer of about 5 mm thickness) and detecting the external surface using the direction of the external auditory canal. Particularly, the surface that has a normal that points in the direction of the external canal (with a deviation within 40 degrees) is considered to be the external surface.

Further, according to an embodiment, annotating of the temporal bone in the image can be performed. Again, the volume used can be a cuboidal volume, particularly a cube, a spherical volume or any other suitable volume.

Further, according to an embodiment of the method, the method further comprises the steps of:
Predicting a location of the internal auditory canal based on the segmented cochlear region of the image corresponding to the cochlear in the image, and
Defining a volume comprising the predicted location, and
Segmenting the internal auditory canal within the volume thereby obtaining a segmented internal auditory canal region of said image M corresponding to the internal auditory canal in the image.

Further, in an embodiment of the invention, annotating the internal auditory canal in the image can be performed. Again, the volume used can be a cuboidal volume, particularly a cube, a spherical volume or any other suitable volume.

Further, according to an embodiment of the method, the method further comprises the steps of:
Predicting a location of the facial nerve based on the segmented cochlear region of the image M corresponding to the cochlea, and the segmented semicircular canal region corresponding to the three semicircular canals in the image,
Defining a volume comprising the predicted location, and
Segmenting the facial nerve within the volume (e.g. with help of a Hessian-based enhancement filter) thereby obtaining a segmented facial nerve region of the image M corresponding to the facial nerve in said image.

Furthermore, according to an embodiment, annotating the facial nerve in the segmented image is performed.

Further, according to an embodiment of the method, the method further comprises the step of graphically visualizing on a display for a user at least one of: the segmented cochlear region, the segmented semicircular canal region, the segmented ossicles region, the segmented tympanic membrane region, the segmented external auditory canal region, the segmented temporal bone region, the segmented internal auditory canal region, the segmented facial nerve region

Further, according to a further aspect of the present invention a system for automatically segmenting the cochlea of a person comprised in an input image M comprised of voxels V each voxel comprising an intensity value being indicative of the intensity of the respective voxel is disclosed, wherein the system comprises at least one processor and a display, the system being adapted to execute the method according to the present invention.

Particularly, the system is configured to display one, several or all of the following segmented regions of the image M on the display: the segmented cochlea region, the segmented semicircular canal region, the segmented ossicles region, the segmented tympanic membrane region, the segmented external auditory canal region, the segmented temporal bone region, the segmented internal auditory canal region, the segmented facial nerve region

Particularly, the respective segmented region can be overlayed onto the image M on the display and/or displayed in an isolated fashion.

According to a further aspect of the present invention a computer program is disclosed, the computer program comprising instructions which, when the program is executed on the system according to the present invention, cause the system to execute the steps of the method according to the present invention.

According to a further aspect of the present invention a computer-readable, particularly non-transitory, storage medium comprising instructions which, when executed by the system according to the invention (or by a computer), cause the system according to the invention (or the computer) to carry out the steps of the method according to the present invention.

Furthermore, yet another aspect of the present invention relates to a data carrier signal carrying the computer program according to the present invention.

Further features and advantages of the present inventions as well as embodiments of the present invention shall be described in the following with reference to the Figures, wherein

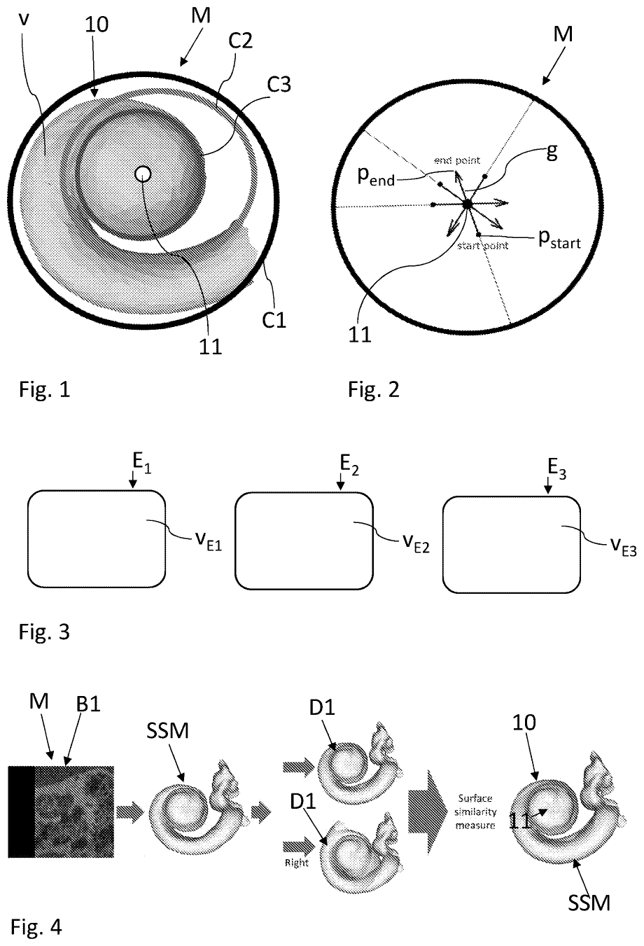

FIG. 1 shows an illustration of the cochlea and its characteristic shape indicated with different circles;

FIG. 2 shows the geometric principle of automatically detecting the center of the cochlea;

FIG. 3 schematically shows an empty first gradient accumulator image E, employed for detecting a center of the cochlea in a medical image;

FIG. 4 shows fitting the SSM of the ear to the cochlea in an image based on a detected center of the cochlea using a surface similarity measure which also allows to detect whether the cochlea belongs to the left or right ear;

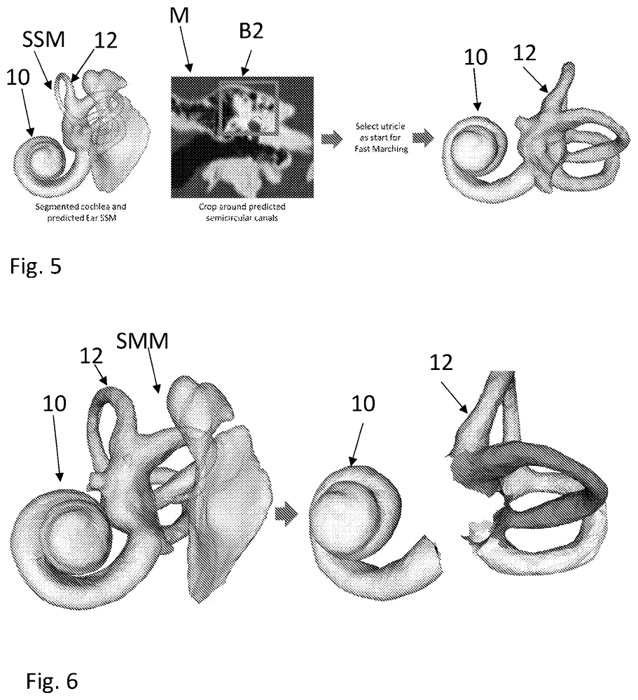

FIG. 5 shows the prediction of the semicircular canals location and their segmentation based on the segmented cochlea;

FIG. 6 illustrates a labeling of the semicircular canals based on the ear SSM adjusted to the segmented cochlea and semicircular canals;

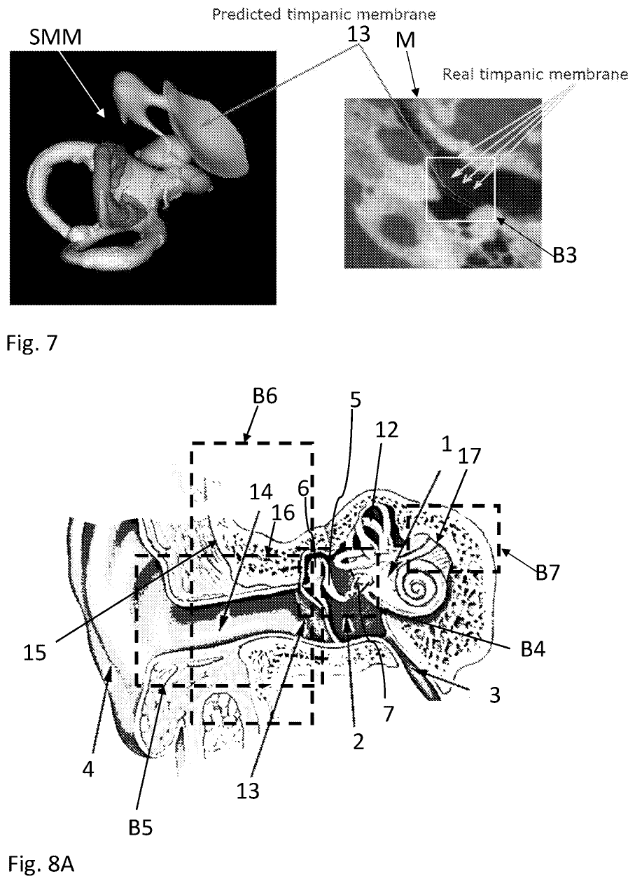

FIG. 7 illustrates predicting the location of the tympanic membrane as well as segmenting the tympanic membrane;

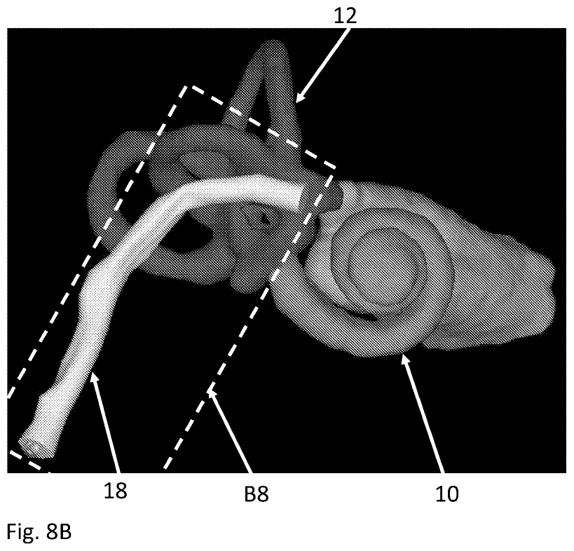

FIG. 8A illustrates e.g. the temporal bone prediction;

FIG. 8B illustrates e.g. the facial nerve prediction;

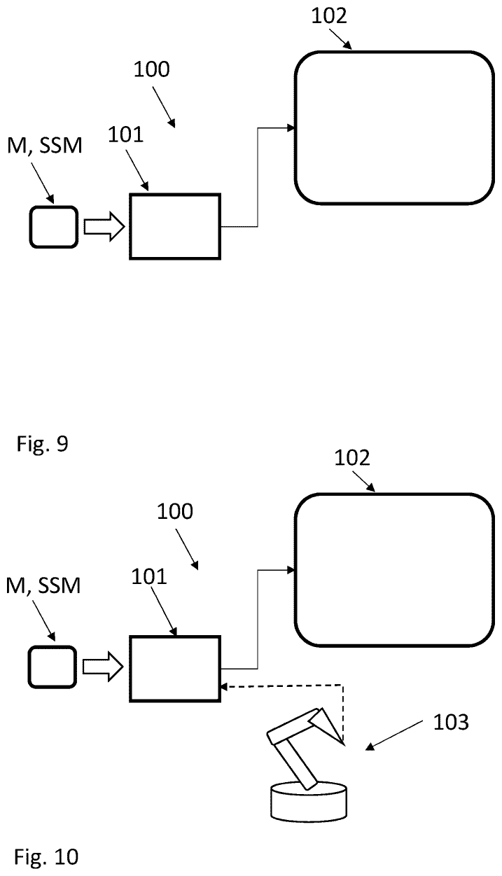

FIG. 9 shows an embodiment of a system according to the present invention; and

FIG. 10 shows an embodiment of a system according to the present invention.

The present invention provides a method for automatically detecting and segmenting the cochlea of a person comprised in an image M comprised of voxels v each voxel comprising an intensity value being indicative of the intensity of the respective voxel v. Such an image can be acquired by a suitable medical imaging device which can be part of the system according to the present invention.

In order to detect the center 11 of the cochlea 10 an empty first gradient accumulator image E₁ is generated as indicated in FIG. 3 that is comprised of voxels v_{E1}, wherein each voxel v_{E1} of the first gradient accumulator image E₁ is associated with a voxel v of the image M. Then, for each voxel v of the image M a gradient vector g pointing in a direction towards a largest possible intensity increase of the image M is computed as well as a first gradient line extending in the direction of the gradient vector g the first gradient line starting at a first start point p_{start,1} and ending at a first end point p_{end,1} (cf. FIG. 2), wherein the intensity value of each voxel v_{E1} of the first gradient accumulator image E₁ whose corresponding voxel v of the image M intersects with the respective first gradient line is increased by a pre-defined constant increment.

Furthermore, an empty second gradient accumulator image E₂ is generated that is comprised of voxels v_{E2} (cf. FIG. 3), each voxel v_{E2} of the second gradient accumulator image E₂ is also associated with a voxel v of the image M, wherein for each voxel v of the image M a gradient vector g pointing in a direction towards a largest possible intensity increase of the image M is computed as well as a second gradient line extending in the opposite direction of the gradient vector g the second gradient line starting at a second start point p_{start,2} and ending at a second end point p_{end,2} (cf. FIG. 2), wherein the intensity value of each voxel v_{E2} of the second gradient accumulator image E₂ whose corresponding voxel v of the image M intersects with the respective second gradient line is increased by a pre-defined constant increment.

Likewise, an empty third gradient accumulator image E₃ comprised of voxels v_{E3} is provided (cf. FIG. 3), each voxel v_{E3} of the third gradient accumulator image E₃ in turn being associated with a voxel v of the image M, wherein for each voxel v of the image M a gradient vector g pointing in a direction towards a largest possible intensity increase of the image M is computed as well as a third gradient line extending in the direction of the gradient vector g the third gradient line starting at a third start point p_{start,3} and ending at a third end point p_{end,3} (cf. FIG. 2), wherein the intensity value of each voxel v_{E3} of the first gradient accumulator image E₃ whose corresponding voxel v of the image M intersects with the respective third gradient line is increased by a pre-defined constant increment.

A cochlea descriptor image E_{descriptor} is then generated comprised of voxels v_{c} by combining the first, second and third gradient accumulator images E₁, E₂, E₃, and selecting a number P≥1 of voxels v_{c} of the cochlea descriptor image E_{descriptor} having intensity values being larger than the intensity values of all other voxels of the cochlea descriptor image E_{descriptor} as predictions of the location of the center of the cochlea in said image M. Particularly, the procedures described above can be employed to determine candidates for the center 11 of the cochlea 10.

The reason for using the three accumulator images E₁, E₂, E₃ is based on the finding that in general the cochlea shape can be described as a snail shape and consists of two and a half turns. A relatively large number of normals of cochlea surface points will therefore pass through the actual center 11 of the cochlea 10.

The main principle behind the cochlea center detection algorithm can therefore be understood as the task to find a point in space that contains maximal number of surface normal intersections.

This principle is illustrated in FIG. 2 for the detection of a circle's center with a known radius. Here, a short line is defined that starts before the circle's center and ends after the circle's center. Such an algorithm permits to detect the circles of the defined size.

This principle can be used to design an algorithm for cochlea center detection, as the cochlea's mean radius and its variation is known.

In the following, as an example, a pseudo code for detecting a circle's center is stated. The algorithm requires the following as input:
An Image M comprising the center to be detected (e.g. center of a cochlea, here for simplicity of a circle), which image M can be a CT, MR or Cone-Bean CT volumetric image that contains the circle/cochlea.
an empty image E, particularly with the same origin and size as M. The image spacing (voxel size) can be different from the image M. This image is called accumulator image.
start_{distance}—distance from voxel location to line start point (along voxel gradient direction).
end_{distance}—distance from voxel location to line end point (along voxel gradient direction),

and yields as output an accumulated gradient vector intersection image.

The corresponding pseudocode can be formulated as:

```
Circle center detection algorithm (M, start_{distance}, end_{distance})
  for each voxel v_{E} in E
    v_{E} = 0; // initialization
  end for
  for each voxel v_{M} in M
    Compute gradient vector: \vec{g} at v_{M}
    $Normalize : \vec{g} = \frac{\vec{g}}{‖ g ‖}$
    Start gradient line point: pₛₜₐᵣₜ = position(v_{M}) + start_{distance} * \vec{g}
    End gradient line point: p_{end} = position(v_{M}) + end_{distance} * \vec{g}
    for each voxel v_{E} in E
      if line (pₛₜₐᵣₜ; p_{end}) intersects v_{E}
        v_{E} = v_{E} + 1
      end if
    end for
  end for
```

The circle center detection algorithm can be easily adapted to the situation of the cochlea described initially, by realizing that the cochlea shape defines three such circles which are schematically shown in FIG. 1.

Particularly, a cochlea can be modeled as said three different circles as follows:

A first circle C1 captures the outer wall of the cochlea's 10 basal turn. Anatomically it represents the changes from cochlea bone (high intensity) to cochlea liquid (low intensity), these gradient vectors point towards the cochlea center 11.

A second circle C2 captures the inner wall of the cochlea's 10 basal turn. Anatomically it represents the changes from cochlea liquid (low intensity) to cochlea bone (high intensity), these gradient vectors point outwards away from the cochlea center 11 but are aligned with the center.

A third circle C3 captures the outer wall of the cochlea's second and second-and-a-half turns. Anatomically it represents the changes from cochlea bone (high intensity) to cochlea liquid (low intensity), these gradient vectors again point towards the cochlea center 11.

In terms of the pseudocode this can be implemented as follows:

Having the input image M, we compute an accumulator image Eᵢ for each circle:

Namely, for circle C1 the accumulator image is:

E₁=Circle Center Detection Algorithm(M,3.0,5.0).

For the second circle C2, the gradient direction is outward, so we use the negative distances:

E₂=Circle Center Detection Algorithm(M,−3.0,−2.0).

For the third circle C3 one can use:

E₃=Circle Center Detection Algorithm(M,0.5,2.0).

The Cochlea descriptor image is then e.g. computed as the multiplication of the 3 accumulated images:

E_{descriptor}=E₁*E₂*E₃

Wherein for instance the brightest voxel of the E_{descriptor} can be chosen as a representative of the center 11 of the cochlea 10. Other procedures for selecting candidates of the center 11 of the cochlea as described herein can also be applied.

A preferred variant of finding candidates for the center 11 of the cochlea 10 is to thresholding the cochlea descriptor image E_{descriptor} with a value obtained by multiplying the brightest voxel of the cochlear descriptor image with a factor in the range from 0.6 to 0.8, preferably 0.7, i.e., only the corresponding brightest fraction of the voxels are considered. This usually results in a number of 3D objects in the descriptor image comprising the highest accumulated densities, wherein each candidate for the center 11 of the cochlea is a center of such an object.

As indicated in FIG. 4, these possible centers of the cochlea can be tested as follows in an embodiment of the invention in order to find the true center 11 of the cochlea 10:
a. Crop around the respective candidate for the center of the cochlea with e.g. a box B1 of size of 20×20×20 mm (other volume shapes and sizes may also be used)
b. Automatically segment tubular structures with the help of Hessian-based enhancement filters in said box B1, wherein preferably the segmented structures is/are converted into a 3D mesh, particularly by applying a marching cube algorithm to the segmented structure, so as to obtain a 3D surface of the segmented structures.
c. Automatically fit the ear statistical shape model (SSM), namely the cochlea part only, into the segmented data D1, particularly by fitting the cochlea part to said 3D surface, wherein the left and the right cochlea ear SSM is fitted as it is initially not known which side (left or right ear) the cochlea belongs to.
d. Based on surface similarity measurements it is automatically determined which candidate represents the center 11 of the cochlea 10 (i.e. results in the best surface similarity). This also yields the side of the cochlea (left or right).

Furthermore, FIG. 5 illustrates the prediction of the semicircular canal's 12 location and their segmentation based on the segmented cochlea 10:
a. In order to automatically predict the location and segment the semicircular canals 12, the ear SSM is automatically adjusted to the segmented cochlea 10, which yields a prediction of the location of the semicircular canals 12.
b. Then a volume such as a box B2 is cropped around the predicted region and
c. finally, a segmentation is automatically performed in the volume to segment the semicircular canals 12 in the image M.

As shown in FIG. 6, by adjusting the ear SSM to the cochlea 10 and semicircular canal 12, the semicircular canals 12 can be labelled (annotated).

Furthermore, the cochlea part and the semicircular canal part of the statistical shape model SSM can be automatically fitted to segmented cochlear 10 and to the segmented semicircular canal 12, based on which locations of the incus 5, malleus 6 and stapes 7 (cf. also FIG. 8A) can be predicted in a volume B4 and then segmented in said volume B4.

Furthermore, once the ear SSM is fitted into cochlea 10 and semicircular canals 12 one also knows an approximate location of the tympanic membrane 13. This is illustrated in an exemplary fashion in FIG. 7. Again, a volume B3 is defined comprising the predicted location, and the tympanic membrane 13 is automatically segmented within the volume B3 thereby obtaining a segmented tympanic membrane region of the image M.

Further, FIG. 8A shows the inner ear 1 and middle ear 2 with the eustachian tube 3 and pinna 4. As illustrated in FIG. 8A, a region of location, an orientation and limit of the external auditory canal 14 can be predicted based on the segmented tympanic membrane 13 (see above). After having defined a volume B5 comprising the predicted region the external auditory canal 14 can be automatically segmented within the volume B5. Based on the segmented external auditory canal 14, a location and orientation of the external surface 15 of the temporal bone 16 can be automatically predicted. After having defined a volume B6 comprising the predicted location, the temporal bone 16 can be automatically segmented within the volume B6 with the help of thresholding (bone always has high intensity). The external surface 15 can be detected using the direction of the external auditory canal 14.

Furthermore, also a location of the internal auditory canal 17 can be automatically predicted based on the segmented cochlear 10, since the inner ear canal 17 is located under the cochlea 10. Having defined a volume B7 comprising the predicted location, the internal auditory canal can be automatically segmented within the volume B7.

Further, a location of the facial nerve 18 can be automatically predicted based on the segmented cochlear 10, and the segmented semicircular canals 12. Having defined a volume B8 comprising the predicted location as shown in FIG. 8B, the facial nerve 18 can be automatically segmented within the volume B8.

The segmented image can be used advantageously used in a variety of different applications such as planning of surgery, monitoring during surgery, image-guided surgery, diagnosis and patient education, particularly showing a planned surgery and/or treatment to a patient, particularly before surgery, etc.

Further, FIG. 9 shows an embodiment of a system 100 according to the present invention configured to automatically detect and segment the cochlea 10 of a human ear comprised in an input image M comprised of voxels v each voxel v comprising an intensity value being indicative of the intensity of the respective voxel. Preferably, the system 100 comprises at least one processor 101 and a display 102, the at least one processor 101 and display 102 being adapted to execute the method according to the present invention.

Preferably, the system is configured to display one, several or all of the following segmented regions of the image M on the display 102: the segmented cochlea region 10 and/or its center 11, the segmented semicircular canal region 12, the segmented ossicles region 5, 6, 7, the segmented tympanic membrane region 13, the segmented external auditory canal region 14, the segmented temporal bone region 16, the segmented internal auditory canal region 17, the segmented facial nerve region 18.

The system 100 preferably is configured to allow input of input data such as the image(s) M and the statistical shape model SSM. The system 100 according to the present invention can be implemented by a general-purpose computer and a display connected thereto, on which computer a computer program can be executed causing the computer to execute the method according to the present invention. However, the system may also comprise or consist of hardwired components/functions configured to execute the method according to the present invention. Furthermore, FIG. 10 shows an embodiment of a system 100 according to the present invention that is tailored towards performing image-guided surgery. Apart from the components described above in conjunction with FIG. 9, the system 100 further comprises at least one surgical instrument 103 for performing surgery on the ear of patient, particularly on one of the segmented parts of the ear such as: the cochlea 10, the semicircular canals 12, the ossicles 5, 6, 7, the tympanic membrane 13, the external auditory canal 14, the temporal bone 16, the internal auditory canal 17, the facial nerve 18. Particularly, the surgical instrument can be driven by an actuator that is configured to be controlled by a physician or fully automatic via an operating device. The operating device may be connected via an interface to the at least one processor/computer 101 and the actuator may be controlled via the at least one processor/computer 101.

Particularly, here, the system 100 is preferably configured to perform one or several or all of the following functions:
Automatically segmenting and particularly annotating at least one structure or multiples structures (such as the cochlea 10, the semicircular canals 12, the ossicles 5, 6, 7, the tympanic membrane 13, the external auditory canal 14, the temporal bone 16, the internal auditory canal 17, the facial nerve 18) of the ear as described herein in at least one image M using a statistical shape model of the ear
Automatically rendering graphical visualizations of said at least one structure or multiple structures (e.g. on display 102);
Perform automatic registration of said at least one structure or of said multiple structures to a current position of the patient,
display of such registered structure(s), particularly in concert with a real-time video representation of the patient during surgery (e.g. on display 102 and/or on a further display)
Automatic tracking of the at least one surgical instrument relative to the patient and relative to the registered structure(s), particularly enabling full visualization of the surgical site.

In the following further aspects of the present invention and embodiments thereof are listed as items. These items may also be formulated as claims of the present invention. The reference numerals in parentheses refer to the Figures described herein.

Item 1: A method for automatically segmenting the cochlea (10) of a person comprised in an image (M) comprised of voxels (v) each voxel comprising an intensity value being indicative of the intensity of the respective voxel (v), the method comprising the steps of:
Providing an empty first gradient accumulator image (E₁) comprised of voxels (v_{E1}), each voxel (v_{E1}) of the first gradient accumulator image (E₁) being associated with a voxel (v) of the image (M), and computing for each voxel (v) of the image (M) a gradient vector (g) pointing in a direction towards a largest possible intensity increase of the image (M) as well as a first gradient line extending in the direction of the gradient vector (g) the first gradient line starting at a first start point (p_{start,1}) and ending at a first end point (p_{end,1}), wherein the intensity value of each voxel (v_{E1}) of the first gradient accumulator image (E₁) whose corresponding voxel (v) of the image (M) intersects with the respective first gradient line is increased by a pre-defined constant increment,
Providing an empty second gradient accumulator image (E₂) comprised of voxels (v_{E2}), each voxel (v_{E2}) of the second gradient accumulator image (E₂) being associated with a voxel (v) of the image (M), and computing for each voxel (v) of the image (M) a gradient vector (g) pointing in a direction towards a largest possible intensity increase of the image (M) as well as a second gradient line extending in the opposite direction of the gradient vector (g) the second gradient line starting at a second start point (p_{start,2}) and ending at a second end point (p_{end,2}), wherein the intensity value of each voxel (v_{E2}) of the second gradient accumulator image (E₂) whose corresponding voxel (v) of the image (M) intersects with the respective second gradient line is increased by a pre-defined constant increment,
Providing an empty third gradient accumulator image (E₃) comprised of voxels (v_{E3}), each voxel (v_{E3}) of the third gradient accumulator image (E₃) being associated with a voxel (v) of the image (M), and computing for each voxel (v) of the image (M) a gradient vector (g) pointing in a direction towards a largest possible intensity increase of the image (M) as well as a third gradient line extending in the direction of the gradient vector (g) the third gradient line starting at a third start point (p_{start,3}) and ending at a third end point (p_{end,3}), wherein the intensity value of each voxel (v_{E3}) of the first gradient accumulator image (E₃) whose corresponding voxel (v) of the image (M) intersects with the respective third gradient line is increased by a pre-defined constant increment, and
forming a cochlea descriptor image (E_{descriptor}) comprised of voxels (v_{c}) using the first, second and third gradient accumulator images (E₁, E₂, E₃), and selecting a number P≥1 of voxels (v_{c}) of the cochlea descriptor image (E_{descriptor}) having intensity values being larger than the intensity values of all other voxels of the cochlea descriptor image (E_{descriptor}) as predictions of the location of the center (11) of the cochlea (10) in said image (M).

Item 2: The method according to item 1, wherein forming the cochlea descriptor image (E_{descriptor}) corresponds to computing the Hadamard product (E₁° E₂° E₃)ᵢⱼ=(E₁)ᵢⱼ(E₂)ᵢⱼ(E₃)ᵢⱼ of the first, second and third gradient accumulator images (E₁, E₂, E₃).

Item 3: The method according to one of the preceding items, wherein the respective gradient line comprises a length extending from the respective start point (p_{start,1}, p_{start,2}, p_{start,3}) to the respective end point (p_{end,1}, p_{end,2}, p_{end,3}), wherein the length of each first gradient line is larger than the length of each third gradient line, and wherein the length of each third gradient line is larger than the length of each second gradient line.

Item 4: The method according to one of the preceding items, wherein the respective first, second and third start point p_{start,1}, p_{start,2}, p_{start,3} is computed according to,

$${p}_{start , i} = position ( v ) + {start}_{i} \cdot g \qquad i = 1 , 2 , 3$$

and wherein the respective first, second and third end point p_{end,1}, p_{end,2}, p_{end,3} is computed according to

$${p}_{end , i} = position ( v ) + {end}_{i} \cdot g \qquad i = 1 , 2 , 3$$

wherein position(v) denotes the position of the receptive voxel (v) of the image (M), and wherein
start₁ is in the range from 2.7 mm to 3.3 mm, wherein particularly start₁ is equal to 3.0 mm,
start₂ is in the range from −3.3 mm to −2.7 mm, wherein particularly start₁ is equal to −3.0 mm,
start₃ is in the range from 0.45 mm to 0.55 mm, wherein particularly start₃ is equal to 0.5 mm,
end₁ is in the range from 4.5 mm to 5.5 mm, wherein particularly end₁ is equal to 5.0 mm,
end₂ is in the range from −1.8 mm to −2.2 mm, wherein particularly end₂ is equal to −2.0 mm,
end₃ is in the range from 1.8 mm to 2.2 mm, wherein particularly end₃ is equal to 2.0 mm.

Item 5: The method according to one of the preceding items, wherein the image (M) is one of: a CT image, an MRI image, a cone-beam CT image.

Item 6: The method according to one of the preceding items, wherein the method further comprises the step of:
Providing a statistical shape model (SSM) of an ear comprising at least a cochlea part corresponding to the cochlea (10) of the ear.

Item 7: The method according to item 6, wherein the method further comprises the step of:
for each voxel (v_{c}) of said number P of voxels:
   defining a volume (B1) in the image (M) having the respective voxel (v_{c}) as a center,
   Segmenting the cochlea (10) within the volume (B1) with help of a Hessian-based enhancement filter thereby obtaining a segmented cochlea region of said image (M), wherein a 3D surface of the cochlea is generated from the segmented cochlea region, particularly by using a marching cube algorithm,
   Fitting of said cochlea part of the statistical shape model (SSM) to the 3D surface,
   Computing a surface similarity measure being indicative of a similarity between a surface of said cochlea part of the statistical shape model (SSM) and the 3D surface,
Determining the voxel (v_{c}) of said number P of voxels as center (11) of the cochlea (10) for which the surface similarity measure fulfils a predefined criterion, wherein particularly the first voxel (v_{c}) among said number P of voxels is chosen as center of the cochlea for which the surface similarity measure drops below a predefined threshold.

Item 8: The method according to item 7, wherein the method further comprises the steps of:
fitting the cochlea part of the statistical shape model (SMM) to the segmented cochlea region using the determined center (11) of the cochlea (10), the statistical shape model (SSM) further comprising a semicircular canal part corresponding to the three semicircular canals (12) of the ear,
Predicting locations of the three semicircular canals (12) in the image (M) as the locations of the three semicircular canals (12) in the semicircular canal part of the statistical shape model (SSM),
Defining a volume (B2) comprising the predicted locations, and
Segmenting the three semicircular canals (12) within the volume (B2) thereby obtaining a segmented semicircular canal region of the image (M).

Item 9: The method according to item 8, wherein the method further comprises the step of:
fitting the cochlea part and the semicircular canal part of the statistical shape model (SSM) to the segmented cochlear region and to the segmented semicircular canal region.

Item 10: The method according to item 9, wherein the method further comprises the steps of:
Predicting locations of the incus (5), malleus (6) and stapes (7) in the image (M) as the locations of incus (5), malleus (6) and stapes (7) in an ossicles part of the statistical shape model (SSM), the ossicles part of the statistical shape model corresponding to incus (5), malleus (6), and stapes (7) of the ear,
Defining a volume (B4) comprising the predicted locations, and
Segmenting the incus, malleus and stapes within the volume thereby obtaining a segmented ossicles region of the image (M) corresponding to incus, mallus and stapes in the image (M).

Item 11: The method according to item 9 or 10, wherein the method further comprises the step of:
Predicting a location of the tympanic membrane (13) as the location of the tympanic membrane (13) in a tympanic membrane part of the statistical shape model (SSM), the tympanic membrane part of the statistical shape model corresponding to the tympanic membrane (13) of the ear,
Defining a volume (B3) comprising the predicted location, and
Segmenting the tympanic membrane (13) within the volume (B3) thereby obtaining a segmented tympanic membrane region of the image (M).

Item 12: The method according to item 11, wherein the method further comprises the steps of:
Predicting a region of location, an orientation and limit of the external auditory canal (14) based on the segmented tympanic membrane region of the image (M),
Defining a volume (B5) comprising the predicted region,
Segmenting the external auditory canal (14) within the volume (B5) thereby obtaining a segmented external auditory canal region of the image (M).

Item 13: The method according to item 12, wherein the method further comprises the steps of:
Predicting a location and orientation of the external surface (15) of the temporal bone (16) based on the segmented external auditory canal region of the image (M)
Defining a volume (B6) comprising the predicted location, and
Segmenting the temporal bone (16) within the volume (B6) with the help of thresholding thereby obtaining a segmented temporal bone region of the image (M), and detecting the external surface using the direction of the external auditory canal (14).

Item 14: The method according to item 7 or one of the items 8 to 13 when referring back to item 7, wherein the method further comprises the steps of:
Predicting a location of the internal auditory canal (17) based on the segmented cochlear region of the image, and
Defining a volume (B7) comprising the predicted location, and
Segmenting the internal auditory canal (17) within the volume (B7) thereby obtaining a segmented internal auditory canal region of said image (M).

Item 15: The method according to items 7, 9 and 12, wherein the method further comprises the steps of:
Predicting a location of the facial nerve (18) based on the segmented cochlear region of the image (M), the segmented semicircular canal region, and the segmented external auditory canal region,
Defining a volume (B8) comprising the predicted location, and
Segmenting the facial nerve (18) within the volume (B8) thereby obtaining a segmented facial nerve region of the image (M).

Item 16: The method according to one of the items 7 to 15, wherein the method further comprises the step of graphically visualizing on a display (102) for a user at least one of: the segmented cochlear region, the segmented semicircular canal region, the segmented ossicles region, the segmented tympanic membrane region, the segmented external auditory canal region, the segmented temporal bone region, the segmented internal auditory canal region, the segmented facial nerve region

Item 17: A system (100) for automatically segmenting the cochlea (10) of a person comprised in an input image (M) comprised of voxels (v) each voxel comprising an intensity value being indicative of the intensity of the respective voxel, wherein the system (100) comprises at least one processor (101) and a display (102), the at least one processor (101) and display (102) being adapted to execute the method according to one of the items 1 to 16.

Item 18: A computer program comprising instructions which, when the program is executed on the system (100) according to item 17, cause the system (100) to execute the steps of the method according to one of the items 1 to 16.

Item 19: A computer-readable storage medium comprising instructions which, when executed by the system according to item 17, cause the system according to claim 17 to carry out the steps of the method of one of the claims 1 to 16.

Item 20: A data carrier signal carrying the computer program of item 18.

1. A method for automatically segmenting the cochlea (10) of a person comprised in an image (M) comprised of voxels (v) each voxel comprising an intensity value being indicative of the intensity of the respective voxel (v), the method comprising the steps of:
Providing an empty first gradient accumulator image (E₁) comprised of voxels (v_{E1}), each voxel (v_{E1}) of the first gradient accumulator image (E₁) being associated with a voxel (v) of the image (M), and computing for each voxel (v) of the image (M) a gradient vector (g) pointing in a direction towards a largest possible intensity increase of the image (M) as well as a first gradient line extending in the direction of the gradient vector (g) the first gradient line starting at a first start point (p_{start,1}) and ending at a first end point (p_{end,1}), wherein the intensity value of each voxel (v_{E1}) of the first gradient accumulator image (E₁) whose corresponding voxel (v) of the image (M) intersects with the respective first gradient line is increased by a pre-defined constant increment,
Providing an empty second gradient accumulator image (E₂) comprised of voxels (v_{E2}), each voxel (v_{E2}) of the second gradient accumulator image (E₂) being associated with a voxel (v) of the image (M), and computing for each voxel (v) of the image (M) a gradient vector (g) pointing in a direction towards a largest possible intensity increase of the image (M) as well as a second gradient line extending in the opposite direction of the gradient vector (g) the second gradient line starting at a second start point (p_{start,2}) and ending at a second end point (p_{end,2}), wherein the intensity value of each voxel (v_{E2}) of the second gradient accumulator image (E₂) whose corresponding voxel (v) of the image (M) intersects with the respective second gradient line is increased by a pre-defined constant increment,
Providing an empty third gradient accumulator image (E₃) comprised of voxels (v_{E3}), each voxel (v_{E3}) of the third gradient accumulator image (E₃) being associated with a voxel (v) of the image (M), and computing for each voxel (v) of the image (M) a gradient vector (g) pointing in a direction towards a largest possible intensity increase of the image (M) as well as a third gradient line extending in the direction of the gradient vector (g) the third gradient line starting at a third start point (p_{start,3}) and ending at a third end point (p_{end,3}), wherein the intensity value of each voxel (v_{E3}) of the first gradient accumulator image (E₃) whose corresponding voxel (v) of the image (M) intersects with the respective third gradient line is increased by a pre-defined constant increment, and
forming a cochlea descriptor image (E_{descriptor}) comprised of voxels (v_{c}) using the first, second and third gradient accumulator images (E₁, E₂, E₃), and selecting a number P≥1 of voxels (v_{c}) of the cochlea descriptor image (E_{descriptor}) having intensity values being larger than the intensity values of all other voxels of the cochlea descriptor image (E_{descriptor}) as predictions of the location of the center (11) of the cochlea (10) in said image (M).

2. The method according to claim 1, wherein forming the cochlea descriptor image (E_{descriptor}) corresponds to computing the Hadamard product (E₁° E₂° E₃)ᵢⱼ=(E₁)ᵢⱼ(E₂)ᵢⱼ(E₃)ᵢⱼ of the first, second and third gradient accumulator images (E₁, E₂, E₃).

3. The method according to one of the preceding claims, wherein the respective gradient line comprises a length extending from the respective start point (p_{start,1}, p_{start,2}, p_{start,3}) to the respective end point (p_{end,1}, p_{end,2}, p_{end,3}), wherein the length of each first gradient line is larger than the length of each third gradient line, and wherein the length of each third gradient line is larger than the length of each second gradient line.

4. The method according to one of the preceding claims, wherein the respective first, second and third start point p_{start,1}, p_{start,2}, p_{start,3} is computed according to,
$${p}_{start , i} = position ( v ) + {start}_{i} \cdot g \qquad i = 1 , 2 , 3$$
and wherein the respective first, second and third end point p_{end,1}, p_{end,2}, p_{end,3} is computed according to
$${p}_{end , i} = position ( v ) + {end}_{i} \cdot g \qquad i = 1 , 2 , 3$$
wherein position(v) denotes the position of the receptive voxel (v) of the image (M), and wherein
start₁ is in the range from 2.7 mm to 3.3 mm, wherein particularly start₁ is equal to 3.0 mm,
start₂ is in the range from −3.3 mm to −2.7 mm, wherein particularly start₁ is equal to −3.0 mm,
start₃ is in the range from 0.45 mm to 0.55 mm, wherein particularly start₃ is equal to 0.5 mm,
end₁ is in the range from 4.5 mm to 5.5 mm, wherein particularly end₁ is equal to 5.0 mm,
end₂ is in the range from −1.8 mm to −2.2 mm, wherein particularly end₂ is equal to −2.0 mm,
end₃ is in the range from 1.8 mm to 2.2 mm, wherein particularly end₃ is equal to 2.0 mm.

5. The method according to one of the preceding claims, wherein the method further comprises the step of:
Providing a statistical shape model (SSM) of an ear comprising at least a cochlea part corresponding to the cochlea (10) of the ear.

6. The method according to claim 5, wherein the method further comprises the step of:
for each voxel (v_{c}) of said number P of voxels:
defining a volume (B1) in the image (M) having the respective voxel (v_{c}) as a center,
Segmenting the cochlea (10) within the volume (B1) with help of a Hessian-based enhancement filter thereby obtaining a segmented cochlea region of said image (M), wherein a 3D surface of the cochlea is generated from the segmented cochlea region, particularly by using a marching cube algorithm,
Fitting of said cochlea part of the statistical shape model (SSM) to the 3D surface,
Computing a surface similarity measure being indicative of a similarity between a surface of said cochlea part of the statistical shape model (SSM) and the 3D surface,
Determining the voxel (v_{c}) of said number P of voxels as center (11) of the cochlea (10) for which the surface similarity measure fulfils a predefined criterion, wherein particularly the first voxel (v_{c}) among said number P of voxels is chosen as center of the cochlea for which the surface similarity measure drops below a predefined threshold.

7. The method according to claim 6, wherein the method further comprises the steps of:
fitting the cochlea part of the statistical shape model (SMM) to the segmented cochlea region using the determined center (11) of the cochlea (10), the statistical shape model (SSM) further comprising a semicircular canal part corresponding to the three semicircular canals (12) of the ear,
Predicting locations of the three semicircular canals (12) in the image (M) as the locations of the three semicircular canals (12) in the semicircular canal part of the statistical shape model (SSM),
Defining a volume (B2) comprising the predicted locations, and
Segmenting the three semicircular canals (12) within the volume (B2) thereby obtaining a segmented semicircular canal region of the image (M).

8. The method according to claim 7, wherein the method further comprises the step of:
fitting the cochlea part and the semicircular canal part of the statistical shape model (SSM) to the segmented cochlear region and to the segmented semicircular canal region.

9. The method according to claim 8, wherein the method further comprises the steps of:
Predicting locations of the incus (5), malleus (6) and stapes (7) in the image (M) as the locations of incus (5), malleus (6) and stapes (7) in an ossicles part of the statistical shape model (SSM), the ossicles part of the statistical shape model corresponding to incus (5), malleus (6), and stapes (7) of the ear,
Defining a volume (B4) comprising the predicted locations, and
Segmenting the incus, malleus and stapes within the volume thereby obtaining a segmented ossicles region of the image (M) corresponding to incus, mallus and stapes in the image (M).

10. The method according to claim 8 or 9, wherein the method further comprises the step of:
Predicting a location of the tympanic membrane (13) as the location of the tympanic membrane (13) in a tympanic membrane part of the statistical shape model (SSM), the tympanic membrane part of the statistical shape model corresponding to the tympanic membrane (13) of the ear,
Defining a volume (B3) comprising the predicted location, and
Segmenting the tympanic membrane (13) within the volume (B3) thereby obtaining a segmented tympanic membrane region of the image (M).

11. The method according to claim 10, wherein the method further comprises the steps of:
Predicting a region of location, an orientation and limit of the external auditory canal (14) based on the segmented tympanic membrane region of the image (M),
Defining a volume (B5) comprising the predicted region,
Segmenting the external auditory canal (14) within the volume (B5) thereby obtaining a segmented external auditory canal region of the image (M).
and wherein the method further comprises the steps of:
Predicting a location and orientation of the external surface (15) of the temporal bone (16) based on the segmented external auditory canal region of the image (M)
Defining a volume (B6) comprising the predicted location, and
Segmenting the temporal bone (16) within the volume (B6) with the help of thresholding thereby obtaining a segmented temporal bone region of the image (M), and detecting the external surface using the direction of the external auditory canal (14).

12. The method according to claim 6 or one of the claims 7 to 11 when referring back to claim 7, wherein the method further comprises the steps of:
Predicting a location of the internal auditory canal (17) based on the segmented cochlear region of the image, and
Defining a volume (B7) comprising the predicted location, and
Segmenting the internal auditory canal (17) within the volume (B7) thereby obtaining a segmented internal auditory canal region of said image (M).

13. The method according to claims 6, 8 and 11, wherein the method further comprises the steps of:
Predicting a location of the facial nerve (18) based on the segmented cochlear region of the image (M), the segmented semicircular canal region, and the segmented external auditory canal region,
Defining a volume (B8) comprising the predicted location, and
Segmenting the facial nerve (18) within the volume (B8) thereby obtaining a segmented facial nerve region of the image (M).

14. The method according to one of the claims 6 to 13, wherein the method further comprises the step of graphically visualizing on a display (102) for a user at least one of: the segmented cochlear region, the segmented semicircular canal region, the segmented ossicles region, the segmented tympanic membrane region, the segmented external auditory canal region, the segmented temporal bone region, the segmented internal auditory canal region, the segmented facial nerve region

15. A system (100) for automatically segmenting the cochlea (10) of a person comprised in an input image (M) comprised of voxels (v) each voxel comprising an intensity value being indicative of the intensity of the respective voxel, wherein the system (100) comprises at least one processor (101) and a display (102), the at least one processor (101) and display (102) being adapted to execute the method according to one of the claims 1 to 14.
